(19) United States
(12) Patent Application Publication
(10) Pub. No.: US 2026/0341473 A1
(43) Pub. Date: Oct. 29, 2026

(54) CLOSED LOOP MONITORING OF STAINING RESULTS WITH SYSTEM CONTROL

(71) Applicant: Ventana Medical Systems, Inc., Tucson, AZ (US)

(72) Inventors: Patrick Scott Brunhoeber, Tucson, AZ (US); David Chafin, Tucson, AZ (US); Michael Otter, Tucson, AZ (US)

(73) Assignee: Ventana Medical Systems, Inc., Tucson, AZ (US)

(21) Appl. No.: 19/702,767

(22) Filed: Jun. 9, 2026

Related U.S. Application Data
(63) Continuation of application No. PCT/US2024/060025, filed on Dec. 13, 2024.
(60) Provisional application No. 63/609,927, filed on Dec. 14, 2023.

(51) Int. Cl. G16H 30/20

(52) U.S. Cl. CPC G16H 30/20

(57) ABSTRACT

A diagnostic system includes a closed-loop monitoring system and a computer with an interface. The system is configured to dispense reagent onto a slide. The slide acts as a platform to support and display tissue samples, cells, and/or other specimens for medical diagnostic processes. The monitoring system is configured to monitor assay procedures and provide feedback on whether the preparation, staining process, and/or analysis on the slide performed as expected. The feedback helps to address workflow touchpoints, variability present in staining environment, dispenser integrity, and/or variations in surface chemistry of the slide. The interface is configured for a user to input commands and/or review feedback.

### CROSS REFERENCE TO RELATED APPLICATIONS

This application is a continuation of International Patent Application Number PCT/US2024/060025, filed Dec. 13, 2024, which is hereby incorporated by reference. International Patent Application Number PCT/US2024/060025, filed Dec. 13, 2024, claims the benefit of U.S. Patent Application No. 63/609,927, filed Dec. 14, 2023, which are hereby incorporated by reference.

### BACKGROUND

In medical diagnostics, ensuring accuracy and reliability is paramount for patient care. In histology, a tissue or other biological sample is typically stained and then analyzed using a microscope. Current staining processes for histology slides are commonly performed in an unmonitored manner in that very little automation is used to determine whether the slides are properly stained. In other words, the staining process is an open loop process in which little to no automatic feedback is provided to ensure the quality of the staining process. In the traditional approach, results are reviewed manually by a trained technician or pathologist. The technician or pathologist visually inspects the stained tissue for typical histological intrinsic landmarks in morphology and cellular features. False-negative staining results are sometimes difficult to diagnose in the absence of reagent application confirmation and/or fluidic integrity checks for the staining process.

Thus, there is a need for improvement in this field.

### SUMMARY

For tissue diagnostics that employ an automated system, controls at the system level (“system controls”) provide clinicians with confidence in the system functionality and ensure patient safety. A system control is typically a mock tissue sample that undergoes a complete assay procedure for verifying the precise application of reagents, activity of reagents, and/or the functionality of automated equipment and fluidics. It was found that current automated systems lacked comprehensive surveillance features such as tracking of instrument performance over time and/or checking for error detection and correction. Consequently, many staining inconsistencies and/or reagent dispensing errors often go undetected unless identified through same slide positive controls. However, the same slide positive controls are labor-intensive processes that come with high costs of procuring and managing control tissue. Additionally, it was discovered that standardized system controls can simplify workflow and provide a greater level of assurance for diagnostic results. For example, standardized system controls can reduce the occurrence of false-negative diagnostic results.

A unique system and method have been developed to address these as well as other issues. In one embodiment, the system includes at least one slide, a closed-loop monitoring system, and a computer with an interface. The slide is adapted as a platform to support and display tissue samples, cells, and/or other specimens for medical diagnostic processes. The monitoring system is configured to monitor assay procedures including but not limited to, tracking instrument performance over time, detecting false-negative results, and/or improving the integrity of results by performing error corrections. The feedback from the monitoring system helps to address workflow touchpoints, variability present in staining environment, dispenser integrity, and/or variations in surface chemistry of the slide. The interface on the computer is configured for a user to input commands and/or review feedback.

According to one version, the monitoring system includes an imaging device, a staining device, and a controller. In one form, the imaging device includes a camera positioned within the staining environment and parallel to the slide. The camera is configured to automatically and/or manually capture images of the slide throughout the diagnostic process, including the staining process. The staining device is configured to highlight specific structures and/or molecules in a tissue sample. The controller typically includes a memory and a processor. The controller is configured to communicate with other parts of the monitoring system. For instance, the controller commands the camera to capture images and the staining device to perform staining. The controller further communicates with the interface of the system to receive user inputs and present feedback. The memory stores an algorithm, such as an artificial intelligence (AI) algorithm, to assist in monitoring the staining process and/or analyzing the stained slides over time. The processor performs calculations, makes decisions, and/or generates commands. According to one example, the algorithm is configured to provide real-time feedback on the integrity of the staining process. Specifically, the algorithm analyzes the captured images to ensure that specific areas on the slide are properly stained and sends real-time data to provide feedback on the integrity of the staining process.

In one embodiment, the slide includes a substrate having a work area or portion and a label area or portion. The work area includes one or more system control areas and at least one tissue placement area. The label area includes a bar code and/or other labeling information. In another embodiment, the work area further includes a tissue control area. In one version, the substrate of the slide includes a plurality of hydrophobic barriers that are positioned proximal to the edges of the substrate to better contain fluids and prevent wicking of fluid off the substrate. In another form, the substrate of the slide includes a plurality of hydrophobic barriers that are positioned across the label area to eliminate the need for over-labeling and/or the use of specialized labels.

The system control areas of the slide include one or more control samples. According to one embodiment, the control samples are tissue samples. According to other examples, control samples are selected for specific tumor types and primary or secondary detection antibodies. The control samples can be presented in diverse formats including but not limited to, cell pellets, tissue micro-arrays, and/or representative sample homogenates. In one example, the system control areas are placed at each corner of the work area of the slide. As should be appreciated, the system control areas can be placed at other locations on the slide. According to one particular embodiment, the system control areas are encapsulated with paraffin for extended shelf life. As should be appreciated, the slide may be made of glass, plastic, and/or other suitable materials.

In one use example, the slide is placed within the diagnostic system that includes the monitoring system. As aforementioned, the monitoring system includes a built-in imaging device, such as the camera, that can continuously monitor multiple slides. As such, the monitoring system ensures that the staining processes are being conducted accurately and consistently. During monitoring of one of the slides, the camera captures a designated area of the slide to verify successful staining. Based on the barcode and/or other information from the labeling portion, the monitoring system receives information such as location and/or chemical composition of the system control areas of the slide. Alternatively or additionally, each image captured by the monitoring system becomes a recorded segment of a qualifying staining run, offering real-time validation of the staining process. The feedback information provided by the closed-loop monitoring system allows for tracking of the system performance and trending of instrument performance over time to indicate changes that require service and/or intervention. By observing changes in staining intensity of the slides over time, valuable capabilities such as enabling predictive analysis for maintenance and/or other system-related interventions can be derived. In this way, not only the reliability of current tests is ensured, but future issues can also be preemptively addressed, bolstering overall performance and reliability of the system.

As should be appreciated, the slides are compatible with other systems, including the systems that do not have an automated surveillance system such as the monitoring system as described.

The systems and techniques as described and illustrated herein concern a number of unique and inventive aspects. Some, but by no means all, of these unique aspects are summarized below.

Aspect 1 generally concerns a system.

Aspect 2 generally concerns the system of any previous aspect including a slide.

Aspect 3 generally concerns the system of any previous aspect in which the slide has a label area.

Aspect 4 generally concerns the system of any previous aspect in which the label area has a barcode.

Aspect 5 generally concerns the system of any previous aspect in which the slide has a work area.

Aspect 6 generally concerns the system of any previous aspect in which the work area has a tissue placement area.

Aspect 7 generally concerns the system of any previous aspect in which the work area has one or more system control areas.

Aspect 8 generally concerns the system of any previous aspect in which the system control areas have one or more control materials.

Aspect 9 generally concerns the system of any previous aspect in which the control materials are selected for specific tumor types and primary or secondary detection antibodies.

Aspect 10 generally concerns the system of any previous aspect in which the control materials are tissue samples.

Aspect 11 generally concerns the system of any previous aspect in which the control materials are configured to facilitate monitoring of assay procedures.

Aspect 12 generally concerns the system of any previous aspect in which the control materials include specific tumor types.

Aspect 13 generally concerns the system of any previous aspect in which the control materials include detection antibodies.

Aspect 14 generally concerns the system of any previous aspect in which the detection antibodies include primary detection antibodies.

Aspect 15 generally concerns the system of any previous aspect in which the detection antibodies include secondary detection antibodies.

Aspect 16 generally concerns the system of any previous aspect in which the control materials are covered with paraffin.

Aspect 17 generally concerns the system of any previous aspect in which the work area has corners.

Aspect 18 generally concerns the system of any previous aspect in which the work area has a rectangular shape.

Aspect 19 generally concerns the system of any previous aspect in which the system control areas are positioned at the corners of the work area.

Aspect 20 generally concerns the system of any previous aspect in which the work area has a tissue control area configured to receive a tissue.

Aspect 21 generally concerns the system of any previous aspect in which the slide includes one or more hydrophobic barriers.

Aspect 22 generally concerns the system of any previous aspect in which the hydrophobic barriers are configured to contain fluids on the slide.

Aspect 23 generally concerns the system of any previous aspect in which the hydrophobic barriers are positioned between the work area and the label area.

Aspect 24 generally concerns the system of any previous aspect including a monitoring system.

Aspect 25 generally concerns the system of any previous aspect in which the monitoring system is configured to execute an algorithm.

Aspect 26 generally concerns the system of any previous aspect in which the algorithm is an artificial intelligence algorithm.

Aspect 27 generally concerns the system of any previous aspect in which the monitoring system includes at least one imaging device.

Aspect 28 generally concerns the system of any previous aspect in which the imaging device is positioned to capture one or more images of the slide.

Aspect 29 generally concerns the system of any previous aspect in which the imaging device includes a camera.

Aspect 30 generally concerns the system of any previous aspect in which the images include the system control areas.

Aspect 31 generally concerns the system of any previous aspect including a dispenser.

Aspect 32 generally concerns the system of any previous aspect in which the dispenser is configured to dispense fluid onto the slide.

Aspect 33 generally concerns the system of any previous aspect in which the fluid includes a reagent.

Aspect 34 generally concerns the system of any previous aspect in which the dispenser is configured to stain a specimen on the slide.

Aspect 35 generally concerns the system of any previous aspect in which the monitoring system includes a controller.

Aspect 36 generally concerns the system of any previous aspect in which the controller is operatively coupled to the dispenser.

Aspect 37 generally concerns the system of any previous aspect in which the controller is operatively coupled to the imaging device.

Aspect 38 generally concerns the system of any previous aspect in which the controller is configured to determine if the slide has been properly stained based on the images of the system control areas of the slide.

Aspect 39 generally concerns the system of any previous aspect in which the controller is configured to determine if the work area has been covered with the reagent.

Aspect 40 generally concerns the system of any previous aspect in which the controller includes a processor.

Aspect 41 generally concerns the system of any previous aspect in which the controller includes memory.

Aspect 42 generally concerns the system of any previous aspect in which the controller is configured to track intra-run variability.

Aspect 43 generally concerns the system of any previous aspect in which the controller is configured to track inter-run variability.

Aspect 44 generally concerns the system of any previous aspect in which the controller is configured to detect fluidic failures.

Aspect 45 generally concerns the system of any previous aspect including a computer.

Aspect 46 generally concerns the system of any previous aspect in which the computer includes an interface.

Aspect 47 generally concerns the system of any previous aspect in which the interface is configured to provide processing information concerning the slide.

Aspect 48 generally concerns the system of any previous aspect in which the computer is operatively coupled to the controller.

Aspect 49 generally concerns the system of any previous aspect in which the system control areas include a dual detection system.

Aspect 50 generally concerns the system of any previous aspect in which the dual detection system is configured to indicate that the slide is properly stained.

Aspect 51 generally concerns the system of any previous aspect in which the dual detection system is configured to indicate that the slide is fully covered by a staining fluid.

Aspect 52 generally concerns the system of any previous aspect in which the dual detection system is configured to facilitate monitoring of cytology staining.

Aspect 53 generally concerns the system of any previous aspect in which the dual detection system is configured to facilitate detection of false negatives.

Aspect 54 generally concerns the system of any previous aspect in which the system control areas include at least two standardized substances that are different.

Aspect 55 generally concerns the system of any previous aspect in which the system control areas include at least two standardized tissue samples that are different.

Aspect 56 generally concerns the system of any previous aspect in which the dual detection system includes a first control and a second control.

Aspect 57 generally concerns the system of any previous aspect in which the first control and the second control are different.

Aspect 58 generally concerns the system of any previous aspect in which the first control and the second control react differently when exposed to the fluid.

Aspect 59 generally concerns the system of any previous aspect in which the first control and the second control produce different colors when exposed to the fluid.

Aspect 60 generally concerns the system of any previous aspect in which the monitoring system is configured to monitor area size of the system control areas that react with the fluid.

Aspect 61 generally concerns the system of any previous aspect in which the monitoring system is configured to monitor color of the control areas that react with the fluid.

Aspect 62 generally concerns the system of any previous aspect in which the monitoring system is configured to monitor intensity of the system control areas that react with the fluid.

Aspect 63 generally concerns the system of any previous aspect in which the monitoring system is configured to monitor density of the system control areas.

Aspect 64 generally concerns the system of any previous aspect in which the slide has a background color.

Aspect 65 generally concerns the system of any previous aspect in which the background color provides a high contrast for the system control areas.

Aspect 66 generally concerns the system of any previous aspect in which the background color is white.

Aspect 67 generally concerns a method.

Aspect 68 generally concerns the method of any previous aspect including capturing one or more images of one or more system control areas of a slide with a camera of a monitoring system.

Aspect 69 generally concerns the method of any previous aspect including determining the slide has not been processed in a correct manner with a controller of the monitoring system.

Aspect 70 generally concerns the method of any previous aspect including providing feedback in response to the determining the slide has not been processed in the correct manner with the monitoring system.

Aspect 71 generally concerns the method of any previous aspect including tracking intra-run variability with the monitoring system.

Aspect 72 generally concerns the method of any previous aspect including tracking inter-run variability with the monitoring system.

Aspect 73 generally concerns the method of any previous aspect in which the determining the slide has not been processed in the correct manner includes detecting fluidic failures.

Aspect 74 generally concerns the method of any previous aspect including monitoring for cytology staining with a dual detection system.

Aspect 75 generally concerns the method of any previous aspect including detecting false negatives with a dual detection system.

Aspect 76 generally concerns a method of manufacturing.

Aspect 77 generally concerns the method of manufacturing of any previous aspect including producing a substrate.

Aspect 78 generally concerns the method of manufacturing of any previous aspect including placing control materials on the substrate.

Aspect 79 generally concerns the method of manufacturing of any previous aspect including encapsulating the control materials for extended shelf life.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a block diagram of a diagnostic system according to one example.

FIG. 2 is an enlarged view of a slide according to one embodiment used in the system of FIG. 1.

FIG. 3 is a top view of another slide and a corresponding scanned image according to another embodiment.

FIG. 4 is a top view of the slide according to one embodiment.

FIG. 5 is a top view of the slide before staining according to one embodiment.

FIG. 6 is a top view of the slide after staining according to one embodiment.

FIG. 7 is a screen rendering of a results window from an interface according to one example.

FIG. 8 is a histogram illustrating system control area size data according to one example.

FIG. 9 is a histogram illustrating stain intensity data according to one example.

FIG. 10 is a flowchart illustrating a method of operating the FIG. 1 system according to one example.

FIG. 11 is an experimental results diagram showing the density of spots for primary antibody (PAB) titration according to one example.

FIG. 12 is an enlarged perspective view of one example of the camera monitoring the slide in the FIG. 1 system.

FIG. 13 is a graph illustrating intra-run variability, fluidic failures, and inter-run variability in the FIG. 1 system according to one example.

FIG. 14 is a top view of a slide that includes a dual detection system according to another embodiment for use in the FIG. 1 system.

FIG. 15 shows the image processing progression of a slide from a scanned image to a processed image of the slide according to one embodiment.

FIG. 16 is a chart illustrating instrument performance of staining slides with the system control dots across multiple runs.

FIG. 17 is a chart showing staining performance for slides with system control dots across multiple runs and instruments as well as for a run where the slides were processed manually.

FIG. 18 is a chart illustrating analysis of the slide with the system control dots after being stained.

FIG. 19 is a top view of a plurality of slides with first control dots and second control dots according to another example.

FIG. 20 is a chart illustrating analysis of ten slides.

### DETAILED DESCRIPTION OF SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

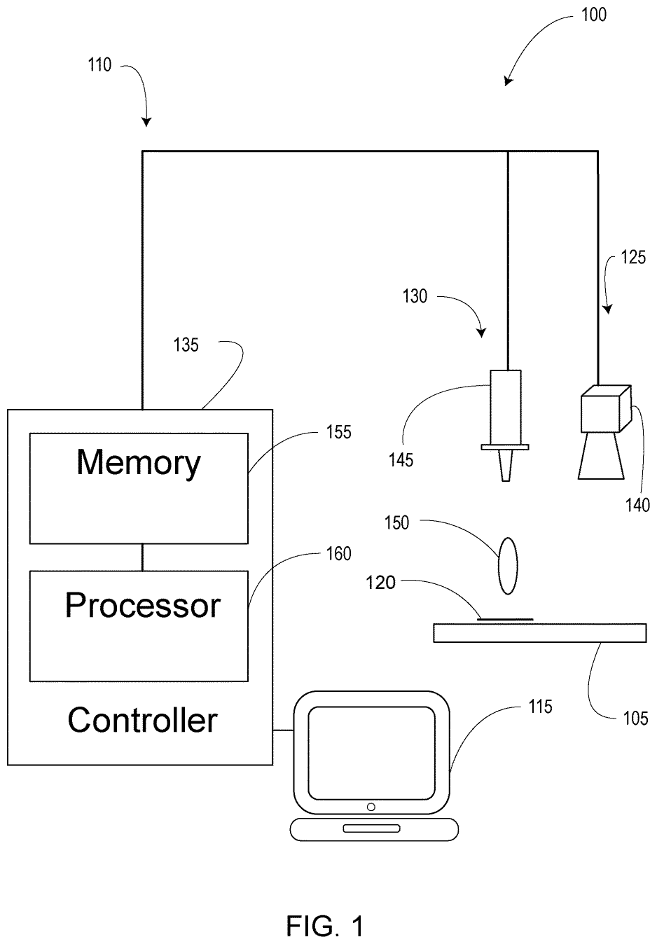

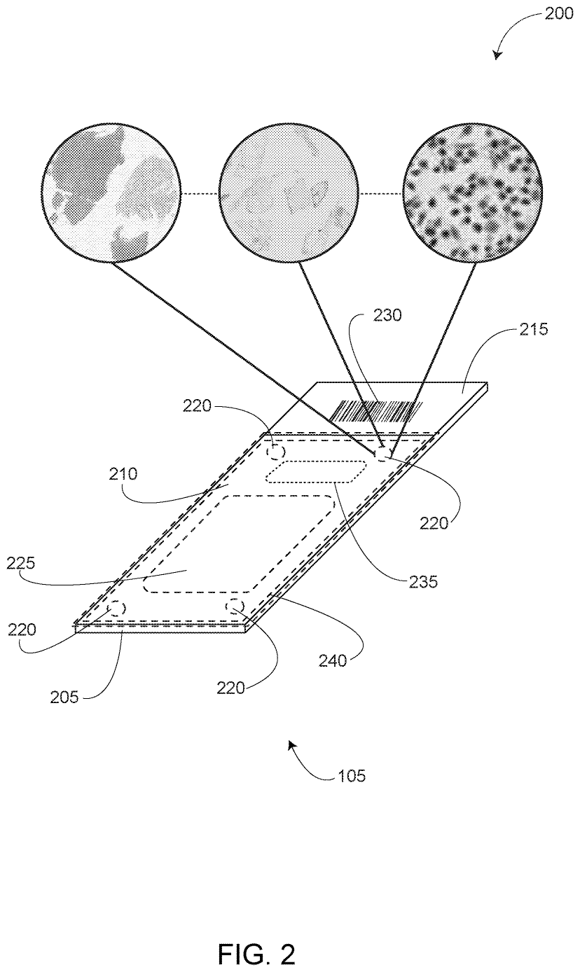

The reference numerals in the following description have been organized to aid the reader in quickly identifying the drawings where various components are first shown. In particular, the drawing in which an element first appears is typically indicated by the left-most digit(s) in the corresponding reference number. For example, an element identified by a “100” series reference numeral will likely first appear in FIG. 1, an element identified by a “200” series reference numeral will likely first appear in FIG. 2, and so on.

FIG. 1 illustrates a diagnostic system 100 according to one embodiment. As shown, the system 100 includes at least one slide 105, a closed-loop monitoring system 110, and a computer 112 with an interface 115. The system 100 is adapted to improve workflow in low to high-throughput medical diagnostic environments and facilitate a comprehensive monitoring and assurance of the entire working process. The slide 105 serves as a platform to support and display samples 120 such as tissue samples, cells, and/or other specimens for assay procedures. The monitoring system 110 is configured to monitor the assay procedures over time and provide feedback on whether preparation, staining process, and/or analysis on the slide 105 performed as expected, improving the integrity of results. The feedback helps to address workflow touchpoints, variability present in staining environments, dispenser integrity, and/or variations in surface chemistry of the slide 105. The computer 112 typically includes a personal computer and is configured to facilitate user inputs and/or feedback reviews. Particularly, the interface 115 on the computer 112 allows clinicians to input various commands, including but not limited to modify settings of the system 100, change area of interest (AOI), and/or move focus point.

In the illustrated example, the monitoring system 110 includes an imaging device 125, a staining device 130, and a controller 135. As should be appreciated, the system 100 may include additional or alternative components. As shown, the imaging device 125 includes a camera 140 positioned within the staining environment and faces the slide 105. The camera 140 is configured to automatically and/or manually capture images of the slide 105 throughout the diagnostic procedures, including the staining process. The images captured include low and/or high-resolution images of the samples 120, bar code, and/or other information presented by the slide 105. The staining device 130 includes a dispenser 145. The dispenser 145 is configured to drop a certain amount of reagent 150 to highlight specific structures and/or molecules in the sample 120. The reagent 150 includes a variety of chemicals and/or dyes suitable for different desired staining effects. As should be appreciated, the choice of the reagent 150 depends on the type of the sample 120 used, the cellular components of interest, and/or the subsequent microscopic analysis to be performed. The controller 135 typically includes a memory 155 and a processor 160. The controller 135 is configured to communicate with other parts of the monitoring system 110. For instance, the controller 135 commands the camera 140 to capture images and/or the dispenser 145 to perform staining. The controller 135 further communicates with the interface 115 of the system 100 to receive user inputs and present feedback. As should be appreciated, the communications within the system 100 may be accomplished through wired and/or wireless communication. The memory 155 stores an algorithm, such as an artificial intelligence (AI) algorithm, to assist in monitoring the staining process and/or analyzing the stained slide 105. The processor 160 serves as a central unit that performs calculations, makes decisions, and/or generates commands to coordinate all activities within the monitoring system 110. According to one example, the algorithm is configured to provide real-time feedback on the integrity of the staining process. Specifically, the algorithm analyzes the captured images to ensure that specific areas on the slide 105 are monitored and sends real-time data to provide feedback on the integrity of the staining process. According to other examples, the algorithms are adapted to auto-correct errors in the staining process, self-recognize patterns within the sample 120, predict outcomes by correlating staining patterns with prognostic data, and/or perform other tasks that enhance accuracy and efficiency of tissue diagnostics.

FIG. 2 illustrates the slide 105 in detail according to one embodiment. As shown, the slide 105 includes a substrate 205 having a work area 210 and a label area 215. The work area 210 includes a plurality of system control areas 220 and a tissue placement area 225. In general, the sample 120 is placed within the tissue placement area 225. The tissue placement area 225 is adapted to interact with various types of reagents 150 to highlight different components of the sample 120. As mentioned before, the tissue placement area 225 may be treated with materials that enhance tissue adhesion and sample integrity on the slide 105. The system control areas 220 can be adapted in diverse formats including, but not limited to, cell pellets, tissue micro-arrays, peptide gels, and/or representative sample homogenates. The system control areas 220 are typically smaller than the tissue placement area 225. In one example, the system control areas 220 are placed at each of the corners of the work area 210 of the slide 105. Therefore, the system control areas 220 do not interfere with the tissue placement area 225. As should be appreciated, the system control areas 220 can be placed at other locations on the slide 105. In certain examples, the system control areas 220 can be located at the tissue placement area 225. In some examples, the system control areas 220 are encapsulated with paraffin for extended shelf life.

The system control areas 220 typically include standardized substances and/or biological specimens that are configured to serve as benchmarks to ensure the staining process and/or other assay procedures are working properly within the system 100. According to one embodiment, the system control areas 220 are adapted to represent specific tumor types and/or primary or secondary detection antibodies. As should be appreciated, the system control areas 220 react in a certain way when exposed to the reagent 150. By analyzing results of the system control areas 220, the clinicians can identify discrepancies that indicate improper staining, reagent failure, and/or system malfunction. In one example, the camera 140 is adapted to automatically capture images of the slide 105. The barcode 230 of the image is adapted to inform the monitoring system 110 of the location and/or composition of the system control areas 220 of the slide 105 that has been scanned. The monitoring system 110 then provides real-time feedback on the quality of the assay procedures. As should be appreciated, the feedback may be presented on the interface 115 of the computer 112 and/or other computerized devices for clinician to review. Alternatively or additionally, the feedback may be sent to the monitoring system 110 for executing feedback control such as self-correcting commands and/or be used for long-term tracking of instrument fluidic performance.

The substrate 205 is typically made of glass but may also be made of and/or combined with other materials. For instance, the substrate 205 may be made of plastic when non-reflectiveness, flexibility, and/or certain fluorescence applications are required. In other examples, the substrate 205, especially the tissue placement area 225, may be coated with polu-L-lysine, treated by silane, positively charged, frosted, and/or other substrate processing techniques to achieve enhanced tissue adhesion, easier marking, and/or facilitate signal detection. According to one example, the color of the substrate 205 is white. The color of the substrate 205 is adapted to better contrast the color changes on the system control areas 220 and/or facilitate the camera 140 to scan the slide 105. As should be appreciated, other color schemes can be used on the substrate 205 in other examples.

The label area 215 includes a barcode 230. The barcode 230 is configured to enhance the precision and efficiency of the system 100 and reduce human errors. For example, the barcode 230 identifies each slide 105 to ensure traceability throughout the diagnostic procedure. During high-volume assay procedures, the barcode 230 allows for automated tracking of the slides 105 through various stages of the staining and/or analysis process. Alternatively or additionally, the barcode 230 encodes information such as the type of the sample 120, the specific control materials used, the time data of preparation, and/or the intended staining protocol. As should be appreciated, other labeling techniques such as quick-response (QR) code and/or textual information may be placed in the label area 215 in addition to or in lieu of the barcode 230.

In a particular embodiment, the work area 210 further includes a tissue control area 235. The tissue control area 235 is configured to provide a direct comparison of a test result with known standards without the need for the monitoring system 110. In a similar manner as the system control areas 220, the tissue control area 235 ensures the reliability of the tissue diagnostic procedure. For instance, the tissue control area 235 typically contains a known tissue and/or cells that react predictably with the reagent 150. By comparing the staining of the tissue control area 235 with expected outcomes, clinicians can confirm that the staining process worked correctly. Further, the efficacy of the reagent 150 can be assessed by the tissue control area 235. For instance, if the reagent 150 has degraded and/or is not otherwise functioning properly, the tissue control area 235 will not react to the reagent 150. Moreover, the tissue control area 235 can provide a benchmark for the interpretation of the actual sample 120 staining patterns, thus helping to distinguish between specific staining and non-specific background staining.

In one version, the substrate 205 of the slide 105 includes a plurality of hydrophobic barriers 240 that are positioned proximal to the edges of the substrate 205 to better contain fluids such as the reagent 150 and prevent wicking of reagent 150 off the substrate 205. In this way, waste of the reagent 150 is reduced and contamination in the system 100 are avoided. In another form, the substrate 205 of the slide 105 includes a plurality of hydrophobic barriers 240 that are positioned across the label area 215 to eliminate the need for over-labeling and/or the use of specialized labels.

As should be appreciated, the slide 105 is compatible with other systems, including the systems that do not have an automated surveillance system such as the monitoring system 110 as described.

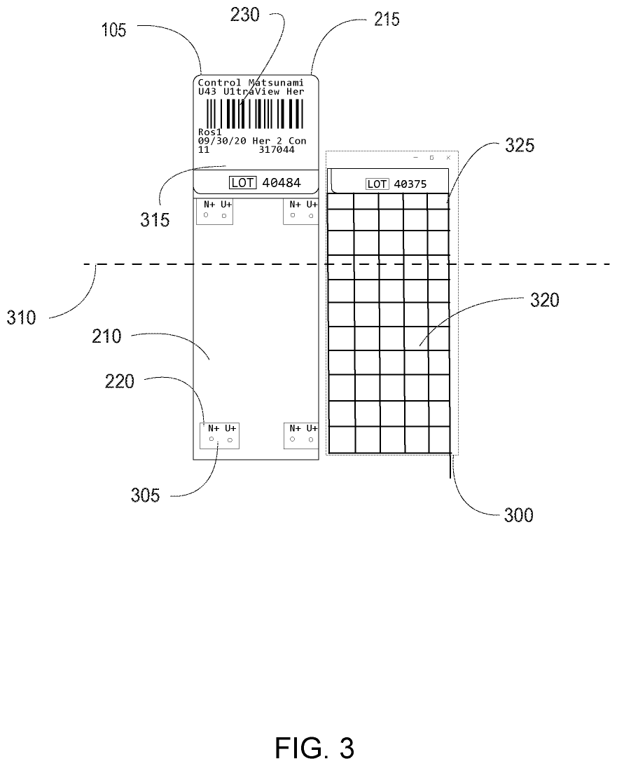

FIG. 3 illustrates the slide 105 and a scanned image 300 of the slide 105 according to one embodiment. As shown, the slide 105 is an immunohistochemical assay integrated with at least four system control areas 220 that are specific to primary and/or secondary antibodies located at four corners of the work area 210. Each of the four system control areas 220 includes at least two control dots or control materials 305 positioned along an edge of the work area 210 that is parallel to a horizontal axis 310. As shown, the control materials 305 are peptide gel dots. As should be appreciated, other materials and/or shapes can be used as the control materials 305 in lieu of the peptide gel dots. In this way, four control materials 305 are positioned along one edge of the work area 210 and four control materials 305 are positioned along the opposing edge of the work area 210. As shown, the label area 215 of the slide 105 includes the barcode 230 and a slide information text 315. The slide information text 315 includes batch number of the slide 105, the type of the sample 120, the specific materials used for the system control areas 220, the date of preparation of the slide 105, and/or the intended staining protocol for this particular slide 105.

The scanned image 300 in FIG. 3 depicts fluidic coverage and homogeneity of the reagent 150 on the slide 105. If the reagent 150 does not adequately cover the work area 210 of the slide 105, the tissue being tested on the slide 105 might in turn not be adequately stained. Inadequate staining of the tissue can lead to incomplete or erroneous test results. Uneven application of the reagent 150 can result in varying intensities across the tissue on the slide 105, which can make tissue analysis more difficult and/or result in misdiagnosis. In the illustrated example, at least two control materials 305 are utilized in each of the system control areas 220 to verify the fluidic coverage and homogeneity of the reagent 150 on the slide 105. In other examples, three or more control materials 305 can be used in each of the system control areas 220. In one variation, full fluidic coverage of the reagent 150 on the slide 105 is confirmed when all eight control materials 305 have reacted with the reagent 150.

During a typical assay procedure of the illustrated example, the primary antibody binds to a target antigen in the tissue sample 120. The secondary antibody, which recognizes the primary antibody, binds to form a complex. An enzyme and/or a dye that is conjugated to the secondary antibody can then be visualized, indicating the presence and location of the target antigen within the tissue sample 120. As shown, a scanned stain 320 on the slide 105 reflects a partial staining. Specifically, the partial stain is observed in the system control area 220 located at the upper-right corner of the work area 210 of the slide 105, as is viewed in FIG. 3. In the illustrated example, the monitoring system 110 indicates a partial staining alert 325 through the scanned image 300 on the interface 115. In another example, the monitoring system 110 notifies the partial staining alert 325 to the other components of the system 100 to attempt to correct the staining error automatically.

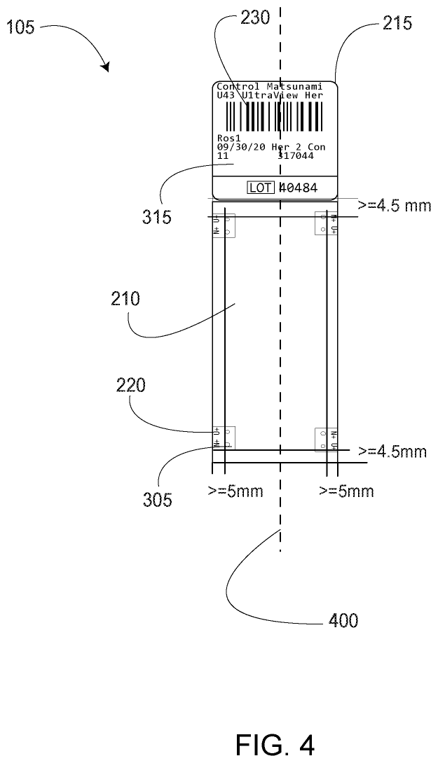

FIG. 4 illustrates a different layout of the system control areas 220 on the slide 105 according to another embodiment. The different layout allows for the staining process that is suitable for certain designs of the system 100 and to monitor fluidic coverage and homogeneity of the reagent 150 on the slide 105. As shown, the slide 105 is an assay integrated with four system control areas 220 located at four corners of the work area 210. Each of the four system control areas 220 includes two control materials 305 positioned along an edge of the work area 210 that is parallel to a vertical axis 400. In this way, four control materials 305 are positioned along one edge of the work area 210 and four control materials 305 are positioned along the opposing edge of the work area 210. As shown, the label area 215 of the slide 105 includes the barcode 230 and a slide information text 315. The slide information text 315 includes batch number of the slide 105, the type of the sample 120, the specific materials used for the system control areas 220, the date of preparation of the slide 105, and/or the intended staining protocol for this particular slide 105.

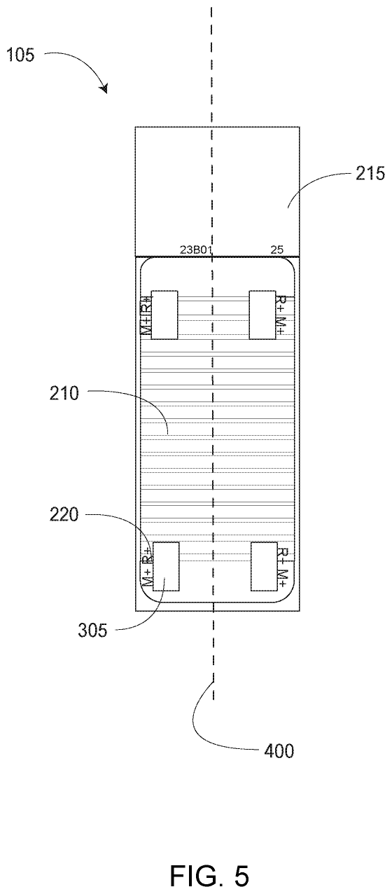

FIG. 5 illustrates the slide 105 before the staining process in one embodiment. As shown, the slide 105 is an assay integrated with four system control areas 220 located at four corners of the work area 210. Each of the four system control areas 220 includes two control materials 305 positioned along an edge of the work area 210 that is parallel to the vertical axis 400. In this way, four control materials 305 are positioned along one edge of the work area 210 and four control materials 305 are positioned along the opposing edge of the work area 210. As shown, the control materials 305 are not visible before reacting with the reagent 150.

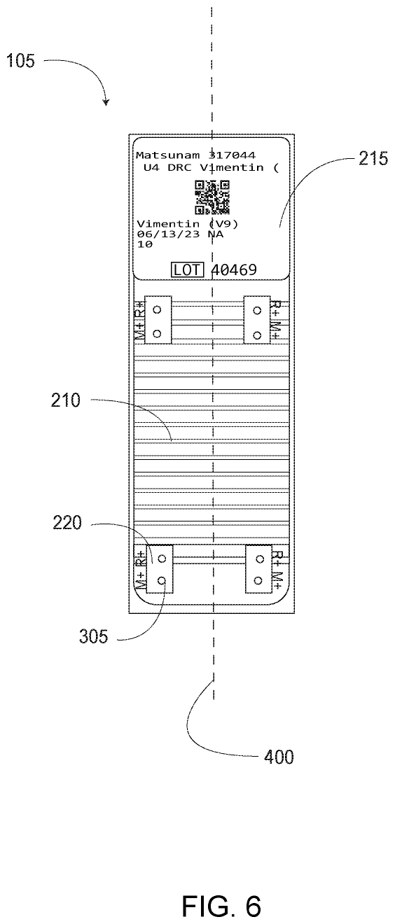

FIG. 6 illustrates the slide 105 after the staining process in one embodiment. As shown, the control materials 305 are visible in a contrasting color against the color of the system control areas 220 after the staining process.

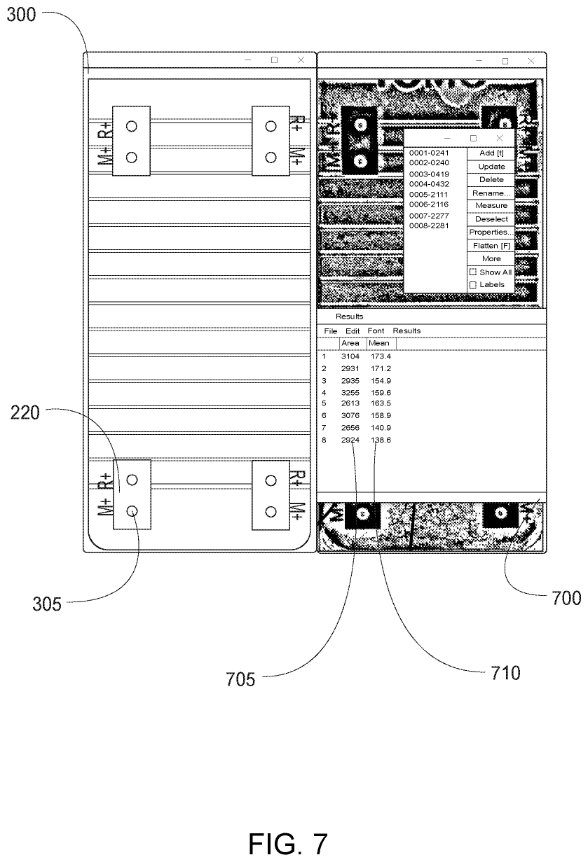

FIG. 7 illustrates a results window 700 showing the results of an automated imaging analysis of the monitoring system 110 after the staining process according to one embodiment. As shown, the results window 700 includes a system control area size data 705 and a stain intensity data 710. As should be appreciated, the results window 700 may include other data such as transmission data. The system control area size data 705 includes a list of area sizes of all of the control materials 305 on the slide 105. The stain intensity data 710 includes a list of mean stain intensity values for each of the control materials 305 corresponding to the system control area size data 705. As shown, the control materials 305 are peptide gel dots having circular shapes. An example command or code for generally automated detection of control materials 305 and calculating the system control area size data 705 and the stain intensity data 710 of the slide 105 having the circular-shaped control materials 305 is provided below:

“open(“C:/Users/example/Desktop/EXAMPLE/Closed Loop/Cabinet Open.jpg”);
selectWindow(“Cabinet Open.jpg”); makeRectangle(30, 1140, 1194, 2478);
run(“Crop”); run(“Duplicate...”, “ ”); run(“8-bit”); run(“Auto Local Threshold”,
“method=Mean radius=50 parameter_1=0 parameter_2=0”); run(“Analyze
Particles...”, “size=1000-Infinity circularity=0.88-1.00 exclude clear include
summarize add”); selectWindow(“Cabinet Open.jpg”); roiManager(“Measure”);”

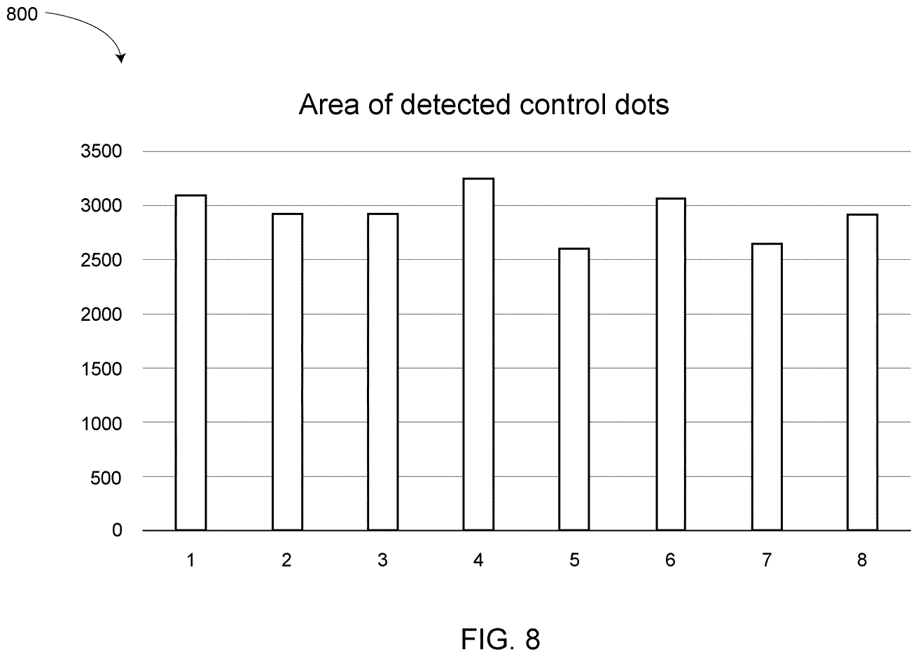

FIG. 8 shows a graphical illustration of the results window 700 according to the embodiment in FIG. 7. As shown, the graphical illustration is a histogram 800 showing the system control area size data 705 derived from the results of the automated imaging analysis of the monitoring system 110 after the staining process. The system control area size data 705 includes a list of area sizes of all of the control materials 305 detected on the slide 105. As should be appreciated, other forms of diagrams and/or charts may be used to show the system control area size data 705.

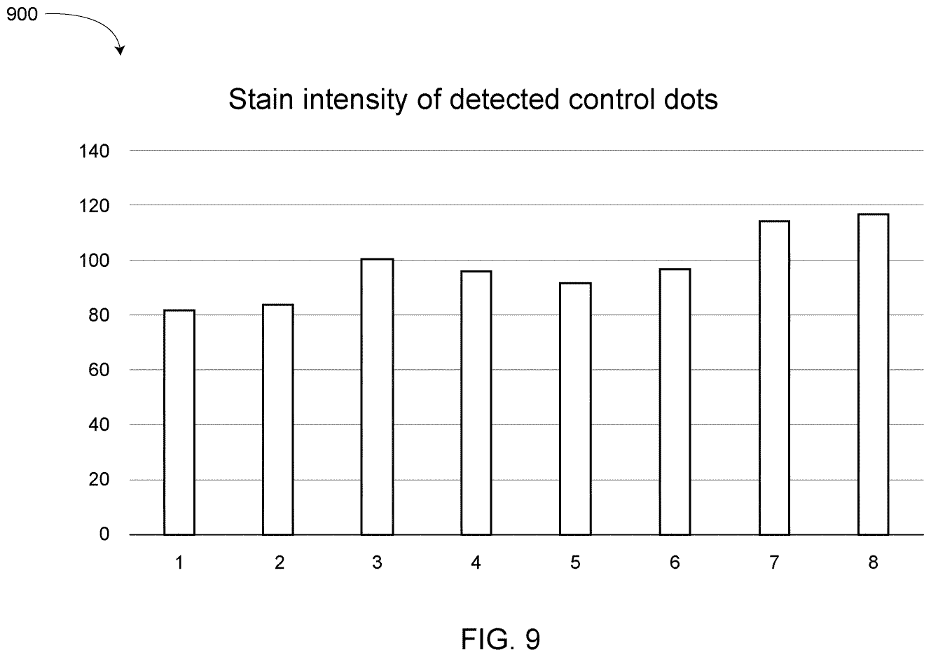

FIG. 9 shows a graphical illustration of the results window 700 according to the embodiment in FIG. 7. As shown, the graphical illustration is a histogram 900 showing the stain intensity data 710 derived from the results of the automated imaging analysis of the monitoring system 110 after the staining process. The stain intensity data 710 includes a list of mean stain intensity values for each of the control materials 305 corresponding to the system control area size data 705. The range of the mean stain intensity values typically falls between 0 to 255. According to one example, the lower the number, the darker the stain is. As should be appreciated, other diagrams and/or charts may be used to show the stain intensity data 710.

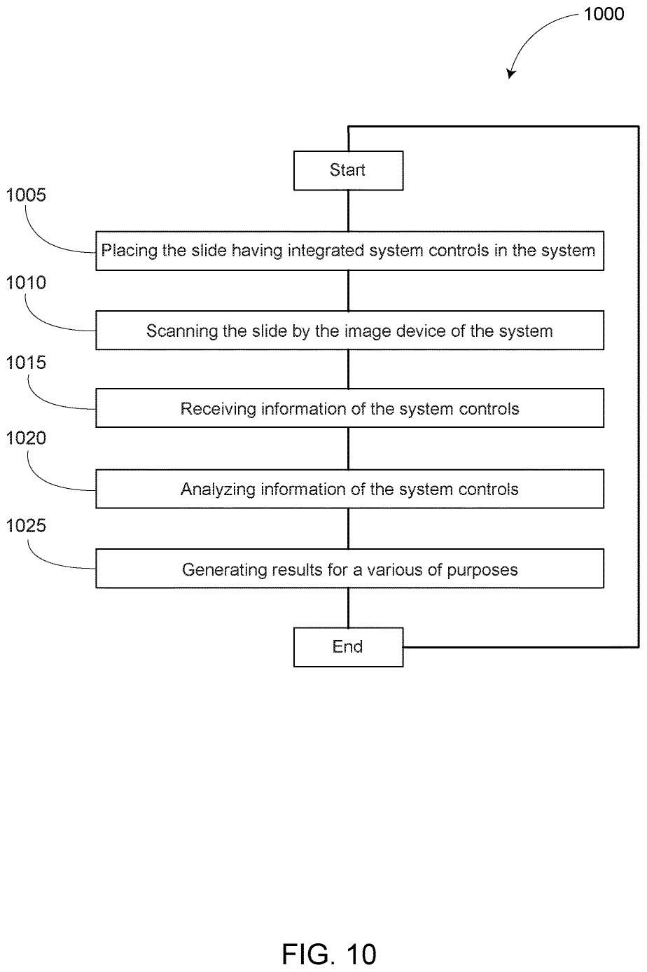

FIG. 10 shows a flowchart 1000 illustrating a use example of the system 100. In stage 1005, the slide 105 is placed within the system 100 having the monitoring system 110. As mentioned before, the monitoring system 110 is configured to automatically provide feedback on whether preparation, staining process, and/or analysis on the slide 105 performed as expected, improving the integrity of the results. In stage 1010, the sample 120, such as the camera 140, continuously monitors a plurality of the slides 105 by scanning the slides 105. According to one example, the camera 140 captures images of a designated area of the slide 105 to verify successful staining while monitoring the slides 105. The images captured includes low and/or high-resolution images of the sample 120, the barcode 230, and/or other information presented by the slide 105. In stage 1015, based on the information from the captured images, the monitoring system 110 receives information of location and chemical composition of the system control areas 220 of the slide 105. The system control areas 220 typically include standardized substances and/or biological specimens that are configured to serve as benchmarks to ensure the staining process and/or other assay procedures are working properly within the system 100.

In stage 1020, the monitoring system 110 analyzes the information received, including the staining results of the system control areas 220, to identify any discrepancies that indicate improper staining, reagent failure, system malfunction, and/or false-negative results. In stage 1025, the monitoring system 110 generates results, including but not limited to, the system control area size data 705 and the stain intensity data 710 of the slide 105 for a variety of purposes. For example, each image captured by the monitoring system 110 becomes a recorded segment of a qualifying staining run, offering real-time validation of the staining process. The feedback information provided by the monitoring system 110 allows for tracking of the performance of the system 100. Over time, by observing changes in the stain intensity data 710 of the slides 105, valuable capabilities such as enabling predictive analysis for maintenance and/or other system-related interventions can be derived. In this way, not only reliability of current tests is ensured, but future issues can also be preemptively addressed, bolstering overall performance and reliability of the system 100.

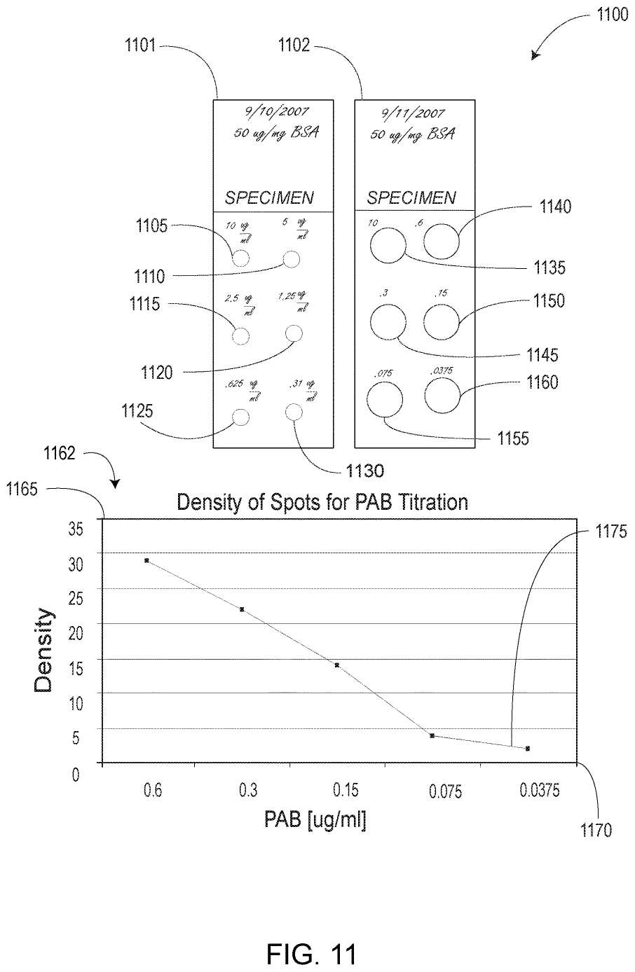

FIG. 11 shows an experimental results diagram 1100 illustrating primary antibody (PAB) titration for determining the optimal concentration of a PAB for use in assays. As can be seen, the experimental results diagram 1100 shows a first slide 1101 and a second slide 1102 containing different dots modified by a bovine serum albumin (BSA) solution that have a concentration of 50 μg/ml. As should be appreciated, the BSA solution increases the chance that the PAB binds only to a target antigen. As illustrated, a series of dilutions of the PAB is prepared. The first slide 1101 has a first area 1105, a second area 1110, a third area 1115, a fourth area 1120, a fifth area 1125, and a sixth area 1130. The first area 1105 is associated with the PAB at 10 μg/ml, and the second area 1110 is associated with the PAB at 5 μg/ml. The third area 1115 is associated with the PAB at 2.5 μg/ml, and the fourth area 1120 is associated with the PAB at 1.25 μg/ml. The fifth area 1125 is associated with the PAB at 0.625 μg/ml, sixth area 1130 is associated with the PAB at 0.31 μg/ml. The second slide 1102 has a seventh area 1135, an eighth area 1140, a ninth area 1145, a tenth area 1150, an eleventh area 1155, and a twelfth area 1160. The seventh area 1135 is associated with the PAB at 10 μg/ml, and the eighth area 1140 is associated with the PAB at 0.6 μg/ml. The ninth area 1145 is associated with the PAB at 0.3 μg/ml, and the tenth area 1150 is associated with the PAB at 0.15 μg/ml. The eleventh area 1155 is associated with the PAB at 0.075 μg/ml, and the twelfth area 1160 is associated with the PAB at 0.0375 μg/ml.

The experimental results diagram 1100 further includes a graph 1162 to visually illustrate the results from the test. As shown, the graph 1162 includes a density-axis 1165, a concentration-axis 1170, and a density-concentration line 1175 indicating the density data derived from the experimental results diagram 1100. The density-concentration line 1175 descends as concentration decreases. In other words, the density-concentration line 1175 indicate that as the concentration of PAB decreases, the density of the spots decreases.

During an example manufacturing process for preparing the slide 105 having a plurality of tissue-free control materials 305, the substrate 205 such as a blank glass strip is provided in the first stage. Next, the substrate 205 is treated with an acid solution, for instance, a 5 N HCl solution, which cleans the substrate 205 by removing impurities and creating a reactive surface, increasing the surface bonding capability with other chemicals. In the next stage, the treated substrate 205 is coated with siloxane, a compound that forms a cross-linked protective layer, further enhancing reactivity and bonding capacity of the substrate 205. Additionally, siloxane treatment is configured to ready the substrate 205 for further chemical reactions. Then, the siloxane-coated substrate 205 is activated using diisocyanate, an additional reactive group that will react with free amines in proteins, such as antibodies or peptides. In the next stage, the activated substrate 205 is exposed to a solution containing a protein. The solution in one example is a BSA solution having a concentration of 50 μg/ml. The protein solution, BSA, binds nonspecific binding sites on the activated substrate 205 to increase the chance that the reagent 150 will bind only to the antigens of interest. The control materials 305 in the system control areas 220 are formed by using BSA to modify areas of interest on the slide 105. The method of applying the protein solution includes, but is not limited to, painting, dropping, and/or printing. In one version, the system control areas 220 on the activated substrate 205 are formed in round shapes. In another version, the system control areas 220 on the activated substrate 205 are formed in stripe shapes. As should be appreciated, the system control areas 220 on the activated substrate 205 may be formed in other shapes. In the final stage, the activated substrate 205 is washed using a solution to form the final ready-to-use slide 105.

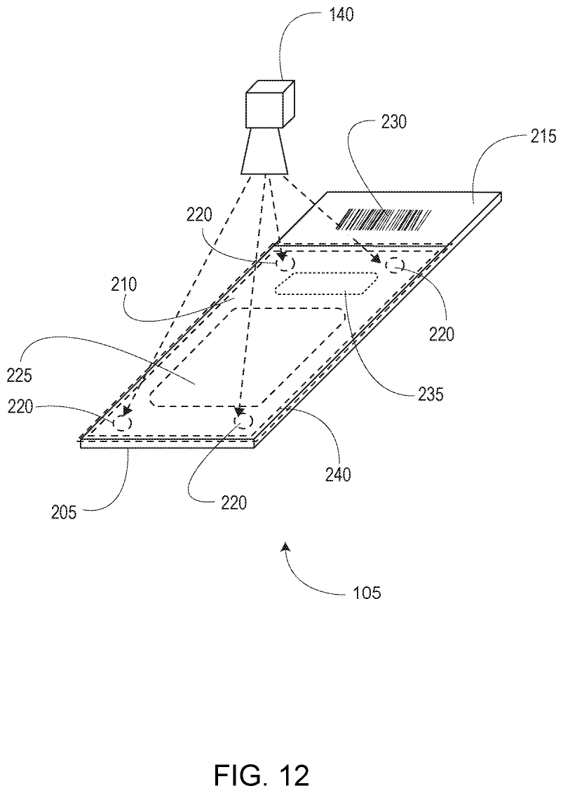

FIG. 12 shows an enlarged perspective view of the camera 140 imaging the slide 105 of the system 100 according to one example. In the illustrated example, the camera 140 faces towards the slide 105. The camera 140 is configured to automatically and/or manually capture images of the slide 105 throughout the diagnostic procedures, including the staining process. The images captured include low and/or high-resolution images of the system control areas 220, the tissue placement area 225, the barcode 230, the tissue control area 235, and/or other information presented by the slide 105. According to one example, the monitoring system 110 is configured to provide real-time feedback on the integrity of the staining process. Specifically, the monitoring system 110 analyzes the captured images to ensure that specific areas on the slide 105 are monitored and sends real-time data to provide feedback on the integrity of the staining process. According to other examples, the monitoring system 110 is adapted to auto-correct errors in the staining process, self-recognize patterns within the system control areas 220, predict outcomes by correlating staining patterns with prognostic data, and/or perform other tasks that enhances accuracy and efficiency of tissue diagnostics. As noted before, the size and/or density of the dots at the system control areas 220 indicate whether the slide 105 was fully and properly covered with the staining reagent 150.

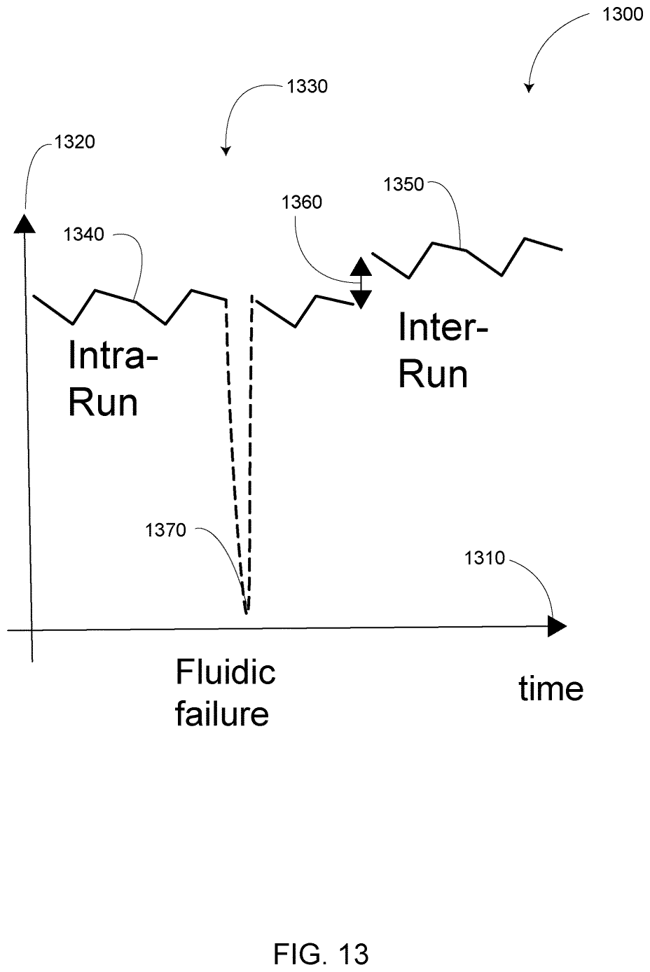

FIG. 13 shows a graph 1300 illustrating a feature of the system 100 that tracks instrument performance over time and/or checks for errors. For instance, the system 100 tracks a plurality of performance indicia, including but not limited to intra-run variability, inter-run variability, and/or fluidic failures. As illustrated, the graph 1300 includes a time axis 1310 that represents time and a control property axis 1320 that represents one or more properties of the system control areas 220 on the slides 105 imaged by the camera 140 after the reagent 150 is applied. For instance, the scanned stain 320 can represent the area of the imaged control dots at the system control areas 220 (see e.g., FIG. 8) and/or the intensity/density of the imaged control dots at the system control areas 220 (see e.g., FIG. 9). In other examples, the control property axis 1320 represents some function or combination of area and intensity of the control dots. The graph 1300 shows fluidic coverage data 1330 in the form of an intra-run line 1340 and an inter-run line 1350.

In one variation, the control property axis 1320 indicates the stain intensity data 710 that is monitored over time by the monitoring system 110 during multiple assay runs. As previously described in FIG. 7, the stain intensity data 710 includes a list of mean stain intensity values for each of the control materials 305 corresponding to the system control area size data 705. According to one embodiment, the stain intensity data 710 is adapted to measure how strongly or weakly the tissue samples 120 are stained, indicating the efficacy of the staining process and identifying any drifts and/or sudden changes in staining quality that might be caused by issues with the reagent 150, the dispenser 145, and/or staining techniques. The fluidic coverage data 1330 is configured to illustrate how effectively and consistently the reagent 150 covers the tissue samples 120 on the slide 105. As should be appreciated, the entire tissue placement area 225 should be uniformly covered with the reagent 150, without any missed areas or uneven application. Variations in the fluidic coverage and/or homogeneity might indicate problems with the dispenser 145 such as blockages in fluidic systems and/or other mechanical issues. In one form, the fluidic coverage data 1330 is quantified by analyzing images of the stained tissue samples 120 captured by the camera 140.

As shown in FIG. 13, the intra-run line 1340 shows fluctuations within a single assay run. The variations in the intra-run line 1340 during this run suggest differences in the stain intensity data 710 and/or the fluidic coverage data 1330 that might occur from one tissue sample 120 to another and/or from one moment to another within the same tissue sample 120. The inter-run line 1350 shows the changes between different assay runs. A gap 1360 between lines indicates a point of transition between runs, and the variations between peaks represent the variabilities from one batch of the slide 105 to another and/or one set of instruments to another. In the illustrated example, a fluidic failure point 1370 indicates a moment where the imaged area and/or intensity of the control dots falls significantly (e.g., below a certain threshold value). The fluidic failure point 1370 in the graph 1300 suggests an issue with the dispenser 145, the reagent 150, and/or other components of the system 100, such as a blockage, leak, and/or other malfunction in the system 100.

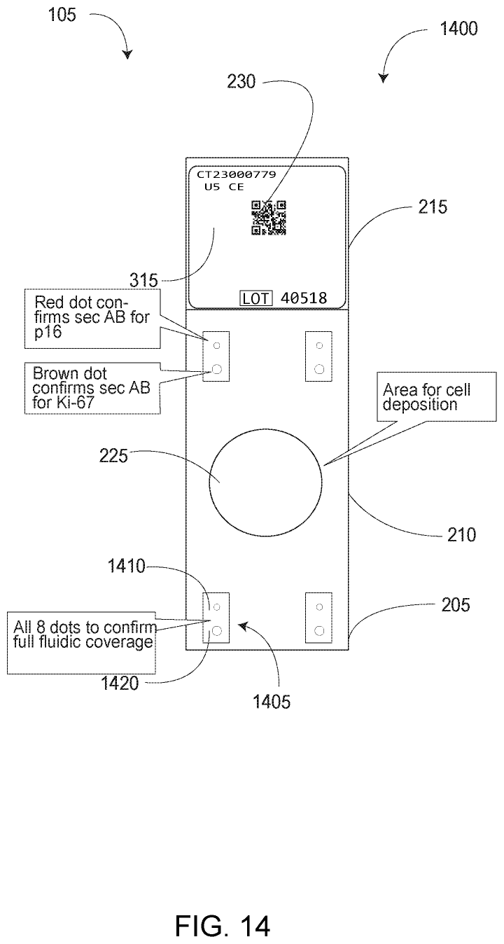

FIG. 14 illustrates a top view of a slide 1400 that includes one or more dual detection systems 1405 according to one embodiment. The slide 1400 in FIG. 14 shares a number of features in common with the slides 105 described above. For the sake of brevity and clarity, these common features will generally not be described in detail again, but please refer to the previous description. As shown, the dual detection system 1405 includes the substrate 205 having the work area 210 and the label area 215. The work area 210 includes the dual detection system 1405 and the tissue placement area 225. In general, the sample 120 is placed within the tissue placement area 225. The tissue placement area 225 is adapted to interact with various types of reagents 150 to highlight different components of the sample 120. As mentioned before, the tissue placement area 225 may be treated with materials that enhance tissue adhesion and sample integrity on the slide 105. The system control areas 220 can be adapted in diverse formats including but not limited to, cell pellets, tissue micro-arrays, peptide gels, and/or representative sample homogenates. The dual detection system 1405 is typically smaller than the tissue placement area 225. In one example, the dual detection systems 1405 are placed at each of the corners of the work area 210 of the slide 105. In this way, the dual detection system 1405 does not interfere with the tissue placement area 225. As should be appreciated, the dual detection system 1405 can be placed at other locations on the slide 105. In some examples, the dual detection systems 1405 are encapsulated with paraffin for extended shelf life.

The dual detection system 1405 typically includes two different standardized substances and/or biological specimens that are configured to serve as benchmarks to ensure the staining process and/or other assay procedures are working properly within the system 100. As should be appreciated, the dual detection system 1405 reacts in a certain way when exposed to the reagent 150. By analyzing results of the dual detection system 1405, the clinicians can identify discrepancies that indicate improper staining, reagent failure, system malfunction, and/or false-negative results. According to one embodiment, the dual detection system 1405 is adapted to represent two specific tumor types and/or primary or secondary detection antibodies.

In the cytology staining example shown, the dual detection system 1405 includes a first control 1410 and a second control 1420. The first control 1410 is configured to indicate the presence of a secondary antibody for p16, a protein commonly used as a biomarker in cancer diagnostics. The first control 1410 turns a first color, such as red, when a secondary antibody that targets p16 has been applied to the slide 105. The second control 1420 is configured to indicate the presence of a secondary antibody that targets Ki-67, a protein that is a marker for cell proliferation. The second control 1420 turns a second color, such as brown, when the secondary antibody specific to Ki-67 has been applied to the slide 105. As the monitoring system 110 tracks the staining processes for a specific diagnostic, if neither the first control 1410 nor the second control 1420 turn colors, a true-negative case is detected. If only the first control 1410 turns red, a false-negative case is detected. As should be appreciated, other sample materials can be used in lieu of the illustrated materials in the first control 1410 and the second control 1420 for other diagnostic purposes. According to one example, when all the first control 1410 and all the second control 1420 react to the reagent 150, full fluidic coverage of the slide 1400 is confirmed by the camera 140 of the system 100.

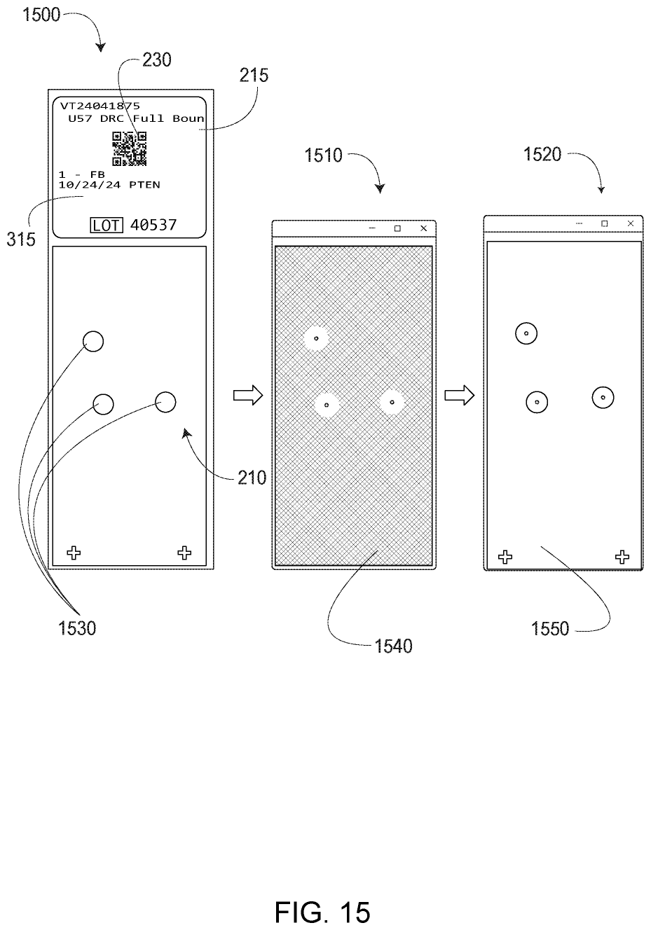

FIG. 15 illustrates the image processing progression of a slide 1500 from a scanned image 1510 to a processed image 1520 of the slide 1500 according to one embodiment. As shown, the slide 1500 is an immunohistochemical assay integrated with three system control dots 1530 that are using a specific recognition peptide for making a “Peptide Gel” spotted onto the slide 1500. In the illustrated example, the system control dots 1530 are PTEN peptides. The system control dots 1530 are positioned in the center area of the work area 210. The system control dots 1530 are configured to be interrogated by certain recognition antibodies. For example, the PTEN peptides are interrogated with the PTEN (SP218) antibody. The slide 1500 with the PTEN peptides is configured to facilitate monitoring certain instrument performance features. As should be appreciated, other configurations of the peptide gel technology can be used to investigate different instrument performance parameters. As shown, the label area 215 of the slide 1500 includes the barcode 230 and the slide information text 315. The slide information text 315 includes, but is not limited to, the batch number of the slide 1500, the specific materials used for the system control dot 1530, the date of preparation of the slide 1500, and/or the intended staining protocol for this particular slide 1500.

After the slide 1500 is stained and scanned, the image of the slide 1500 is analyzed via a segmentation algorithm of the system 100 as the scanned image 1510 illustrates. The segmentation algorithm is configured to automatically select areas of interest. The system 100 then applies the areas of interest to the original image of the slide 1500, resulting in the cropped processed image 1520 as illustrated. Thereafter, the system 100 measures the intensities of the three system control dots 1530 on the processed image 1520.

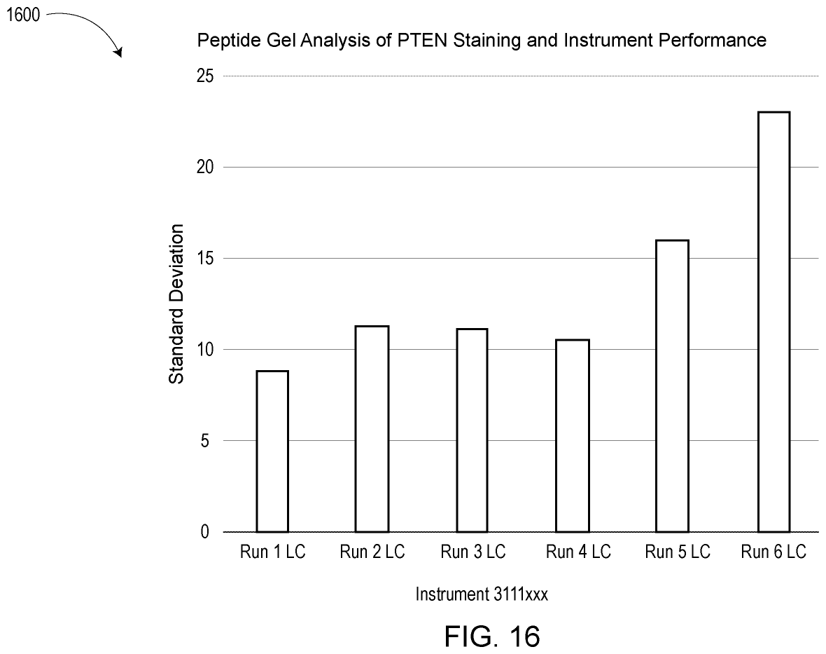

FIG. 16 shows a chart 1600 illustrating instrument performance of staining slides 1500 with the system control dots 1530 across multiple runs. In the illustrated example, the system control dots 1530 are PTEN peptides and are stained with the PTEN (SP218) antibody. The system 100 runs measurements of multiple slides 1500 on the instrument. The chart 1600 in FIG. 16 shows the results from such a test. The measurements of multiple runs can then be compared by intensities and standard deviations between multiple peptide gel dots and multiple slides. In one particular version, the system 100 compares the intensities and standard deviations between multiple system control dots 1530 and multiple slides 1500. As shown in FIG. 16, there is an increase in the standard deviation between the system control dots 1530 in runs 5 and 6 in the illustrated example.

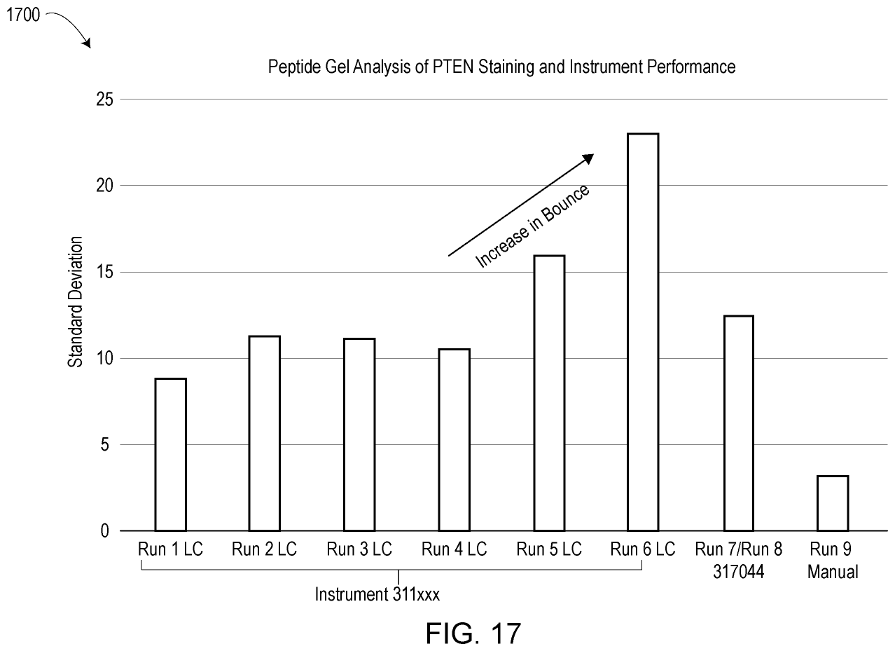

FIG. 17 is a chart 1700 showing staining performance for slides 1500 with system control dots 1530 across multiple runs and instruments as well as for a run where the slides 1500 were processed manually. As can be seen, runs 1-6 were performed on one instrument, and runs 7 and 8 were performed on a different instrument. As shown in the chart 1700, runs 7 and 8 show similar levels of variation. Run 9 was performed as a manual processing run to establish that the variation of the system control dots 1530 (e.g., peptide gels) was not contributing to the observed instrument variation.

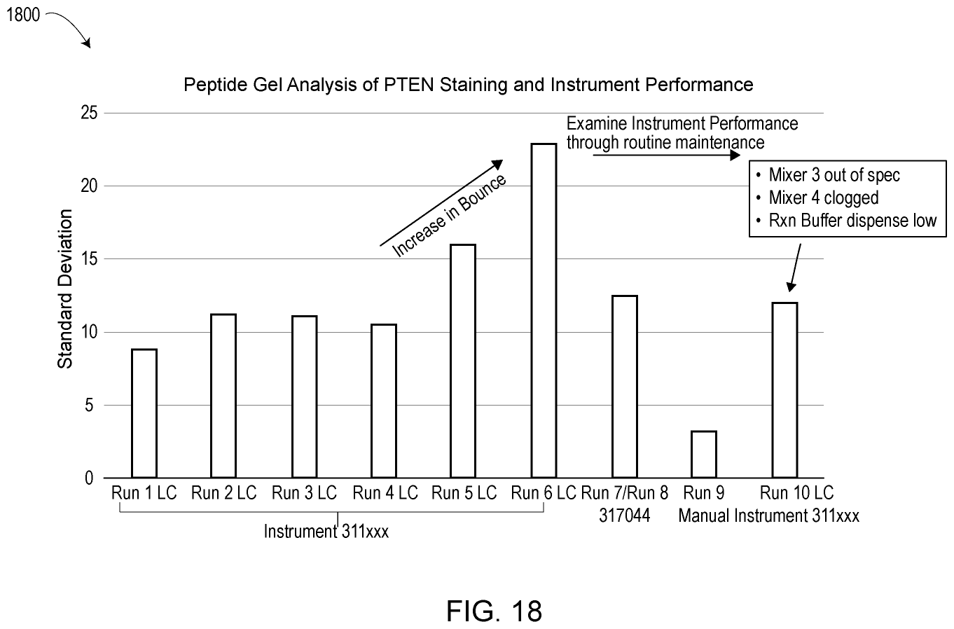

FIG. 18 shows a chart 1800 that expands upon the chart 1700 in FIG. 17. As shown in both the chart 1700 of FIG. 17 and the chart 1800 of FIG. 18, there was an increase in bounce or standard deviation in the later runs (i.e., runs 5 and 6) of the first instrument. In one example, the system 100 is used to perform instrument maintenance on the first instrument to determine the various factors that were compromising optimal performance during staining. Run 10 in the chart 1800 of FIG. 18 shows the results from a run (i.e., run 10) after routine maintenance was performed on the first instrument. In this illustrated example, it was found during maintenance that one of the mixers (mixer 3) was running outside of specification and another mixer (mixer 4) was clogged. It was also found that the buffer dispensing was low. After maintenance was performed to fix these issues, run 10 in the chart 1800 shows that the first instrument was restored to have a similar variation as the earlier runs (i.e., runs 1-4) for the instrument.

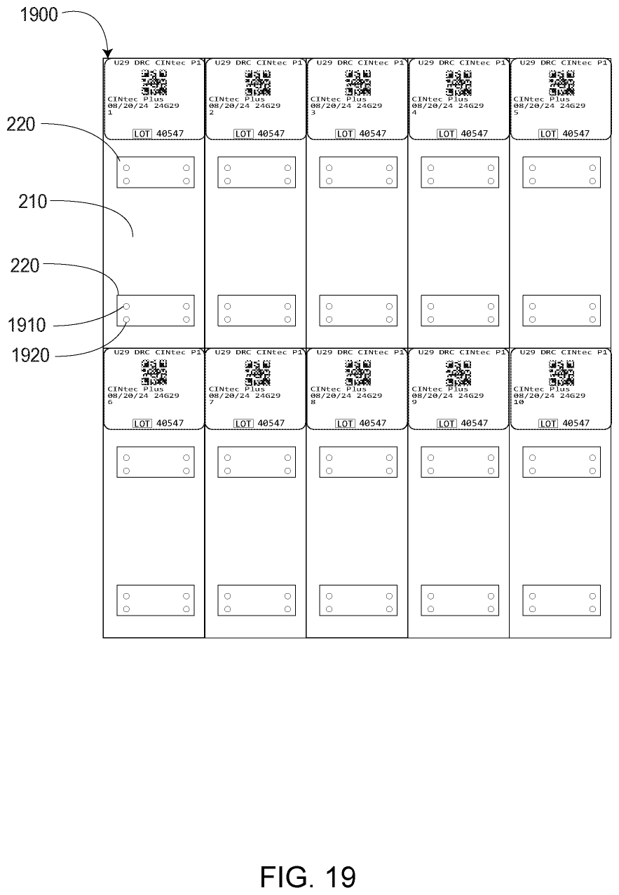

FIG. 19 shows another configuration of control slides 1900 with each having one or more first control dots 1910 and one or more second control dots 1920. Like in the other examples, the slide 1900 includes the work area 210 and the system control areas 220. Each of the system control areas 220 includes at least one first control dot 1910 and at least one second control dot 1920. In the illustrated example, the system control areas 220 are positioned on opposing sides of the work area 210. The first control dots 1910 and the second control dots 1920 in one form are peptide dots that are specific for the detection of antibodies. In some cases, the first control dots 1910 and the second control dots 1920 can have different colors. For instance, the first control dot 1910 can be red, and the second control dot 1920 can be brown. In one test, the slides 1900 with the first control dots 1910 and the second control dots 1920 were analyzed for repeatability over multiple slides 1900 in the same instrument run.

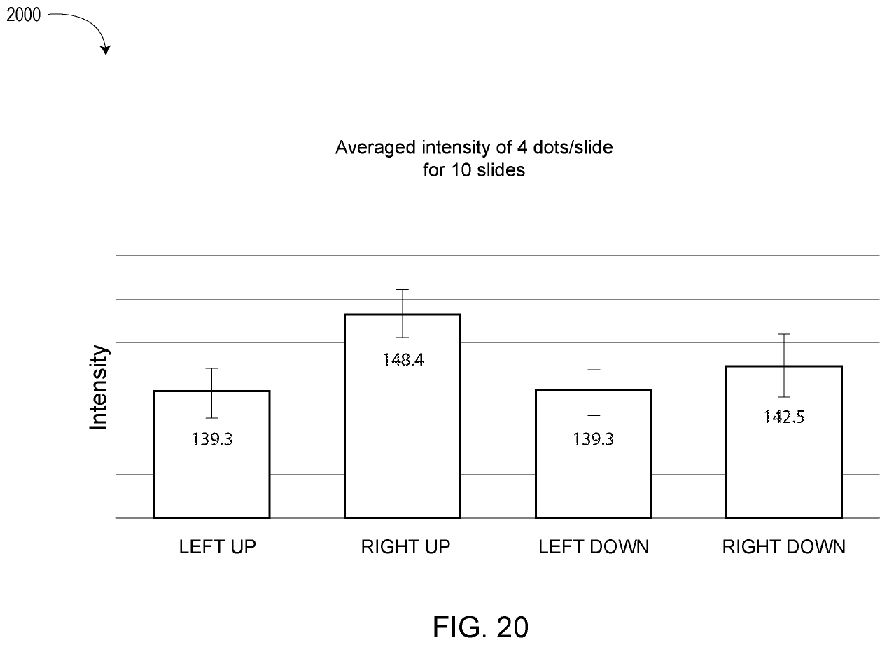

FIG. 20 shows a chart 2000 illustrating an analysis of ten slides 1900. Each of the ten slides contains four system control areas 220. In one test example, the system 100 performs segmentation and image analysis for each of the four second control dots 1920, such as peptide gel dots. Thereafter, the system 100 compares the results for all the ten slides 1900 and observes the total stain variation for all four second control dots 1920 of the ten slides 1900. In one particular test, segmentation and image analysis of each of the brown stained peptide gel dots (with DAB) were analyzed by position and compared for 10 slides 1900. As shown in the chart 2000 of FIG. 20, the total stain variation for all 4 dots for all 10 slides 1900 was 3% (and comparable to the manual run variability demonstrated in the earlier experiment, run 9). It should be recognized that this high degree of reproducibility demonstrates a robust framework for analysis and tracking of instrument performance.

### GLOSSARY OF TERMS

The language used in the claims and specification is to only have its plain and ordinary meaning, except as explicitly defined below. The words in these definitions are to only have their plain and ordinary meaning. Such plain and ordinary meaning is inclusive of all consistent dictionary definitions from the most recently published Webster's dictionaries and Random House dictionaries. As used in the specification and claims, the following definitions apply to these terms and common variations thereof identified below.

“And/Or” generally refers to a grammatical conjunction indicating that one or more of the cases it connects may occur. For instance, it can indicate that either or both of the two stated cases can occur. In general, “and/or” includes any combination of the listed collection. For example, “X, Y, and/or Z” encompasses: any one letter individually (e.g., {X}, {Y}, {Z}); any combination of two of the letters (e.g., {X, Y}, {X, Z}, {Y, Z}); and all three letters (e.g., {X, Y, Z}). Such combinations may include other unlisted elements as well.

“Artificial intelligence” or “AI” generally refers to the ability of machines to perceive, synthesize, and/or infer information. AI may enable a machine to perform tasks which normally require human intelligence. For example, AI may be configured for speech recognition, visual perception, decision making, language interpretation, logical reasoning, and/or moving objects. Typically, AI is embodied as a model of one or more systems that are relevant to tasks that a machine is configured to perform. AI models may be implemented on a device, such as a mechanical machine, an electrical circuit, and/or a computer. AI models may be implemented in an analog or digital form and may be implemented on hardware or software. The implementation of AI may also utilize multiple devices which may be connected in a network.

“Artificial Intelligence Model” or “AI Model” generally refers to a technology or architecture that integrates advanced features, often incorporating artificial intelligence, deep learning, automation, and/or data analytics, to improve performance, efficiency, and user experience. AI models are designed to adapt, learn, and/or optimize behaviors based on the data collected and analyzed, enabling the AI models to make more informed decisions and better support users. AI models are used in various applications, including robotic facilities, smart factories, smart warehouses, smart homes, smart cities, autonomous transportation, smart energy management, and/or smart healthcare among others.

“Barcode” generally refers to a visible arrangement of shapes, colors, lines, dots, or symbols fixed in some medium and arranged on the medium in a pattern configured to encode data. Examples include optical machine-readable representations of data relating to an object to which the barcode is attached such as a Universal Product Code (UPC), or any visible patterns related to any type of Automatic Identification and Data Capture (AIDC) system. Another example of a barcode is a Quick Response Code (QR Code) which arranges various light and dark shapes to encode data. Any suitable medium is envisioned. Examples include an adhesive label, a physical page, a display device configured to display the barcode, or any other object such as a box, a machine, or other physical structure to which the barcode is affixed or upon which it is printed. For example, a barcode may be etched into metal, machined into plastic, or formed by organizing visible three-dimensional shapes into a pattern. The barcode may not be visible to humans but may be fixed using a substance or device that allows the barcode to be visible to sensors in a machine configured to read wavelengths of light outside those detectable by the human eye. Examples of this type of barcode include barcodes printed with ink that is only visible under ultraviolet (i.e., “black”) light, or barcodes displayed using infrared light.

“Camera” generally refers to a device that records visual images. Typically, a camera may record two- and/or three-dimensional images. In some examples, images are recorded in the form of film, photographs, image signals, and/or video signals. A camera may include one or more lenses or other devices that focus light onto a light-sensitive surface, for example a digital light sensor or photographic film. The light-sensitive surface may react to and be capable of capturing visible light or other types of light, such as infrared (IR) and/or ultraviolet (UV) light.

“Computer” generally refers to any computing device configured to compute a result from any number of input values or variables. A computer may include a processor for performing calculations to process input or output. A computer may include a memory for storing values to be processed by the processor, or for storing the results of previous processing. A computer may also be configured to accept input and output from a wide array of input and output devices for receiving or sending values. Such devices include other computers, keyboards, mice, visual displays, printers, industrial equipment, and systems or machinery of all types and sizes. For example, a computer can control a network interface to perform various network communications upon request. A computer may be a single, physical, computing device such as a desktop computer, a laptop computer, or may be composed of multiple devices of the same type such as a group of servers operating as one device in a networked cluster, or a heterogeneous combination of different computing devices operating as one computer and linked together by a communication network. A computer may include one or more physical processors or other computing devices or circuitry and may also include any suitable type of memory. A computer may also be a virtual computing platform having an unknown or fluctuating number of physical processors and memories or memory devices. A computer may thus be physically located in one geographical location or physically spread across several widely scattered locations with multiple processors linked together by a communication network to operate as a single computer. The concept of “computer” and “processor” within a computer or computing device also encompasses any such processor or computing device serving to make calculations or comparisons as part of a disclosed system. Processing operations related to threshold comparisons, rules comparisons, calculations, and the like occurring in a computer may occur, for example, on separate servers, the same server with separate processors, or on a virtual computing environment having an unknown number of physical processors as described above.

“Controller” generally refers to a device, using mechanical, hydraulic, pneumatic electronic techniques, and/or a microprocessor or computer, which monitors and physically alters the operating conditions of a given dynamical system. For example, the controller may be configured to control the behavior of another mechanical and/or electronic device. A controller may include a “control circuit” configured to provide signals or other electrical impulses that may be received and interpreted by the controlled device to indicate how the controlled device should behave. A controller may include a processor for performing calculations to process input or output. A controller may include a memory for storing values to be processed by the processor, or for storing the results of previous processing. A controller may also be configured to accept input and output from a wide array of input and output devices for receiving or sending values. A controller may also be a virtual computing platform having an unknown or fluctuating number of physical processors and memories or memory devices. A controller may thus be physically located in one geographical location or physically spread across several widely scattered locations with multiple processors linked together by a communication network to operate as a single controller. Multiple controllers or computing devices may be configured to communicate with one another or with other devices over wired or wireless communication links to form a network.

“Horizontal” generally refers to a plane and/or direction, which is parallel with the plane of the horizon. In another example, the horizontal plane and/or direction is at a right angle to a vertical plane or direction. An item that moves in the sideways (left to right) direction is generally said to move horizontally. For example, a lever fixed on one end to a rod that is able to move to the left and right is said to move horizontally. In yet another example, the slope of a horizontal line is 0.

“Image” generally refers to a visual representation. The visual representation can for example be of an object, scene, person, and/or abstraction. The image can be in the form of a static picture or can include multiple images in the form of a dynamic video showing motion.

“Interface” or “Human-Machine Interaction (HMI)” generally refers to a computer, a smartphone, a tablet, and other computerized device or system where a user receives information and/or transmits commands. For instance, the interface can be a mechanism through which users can input information or commands and receive feedback or output from a system. In one example, the interface can be visual, such as a graphical user interface (GUI) displayed on a screen. In another example, the interface can be physical, such as buttons, switches, or knobs on a control panel. In a further example, the interface can be auditory, such as spoken commands and feedback, or haptic, such as vibrations or tactile feedback.

“Memory” generally refers to any storage system or device configured to retain data or information. Each memory may include one or more types of solid-state electronic memory, magnetic memory, or optical memory, just to name a few. By way of non-limiting example, each memory may include solid-state electronic Random Access Memory (RAM), Sequentially Accessible Memory (SAM) (such as the First-In, First-Out (FIFO) variety or the Last-In-First-Out (LIFO) variety), Programmable Read Only Memory (PROM), Electronically Programmable Read Only Memory (EPROM), or Electrically Erasable Programmable Read Only Memory (EEPROM); an optical disc memory (such as a DVD or CD ROM); a magnetically encoded hard disc, floppy disc, tape, or cartridge media; or a combination of any of these memory types. Also, each memory may be volatile, nonvolatile, or a hybrid combination of volatile and nonvolatile varieties.

“Neural Network” or “Artificial Neural Network” generally refers to a model composed of multiple nodes. Each node receives a signal from one or more inputs or other nodes. Each node may also perform an operation on the received signal. Each node then sends a signal to one or more other nodes or outputs. The nodes may be arranged in layers such that one or more signals travels across the layers sequentially. The neural network may be given data that trains the neural network. The neural network may be trained to perform a variety of tasks, for example to recognize objects in an image, recognize patterns in a sequence, replicate motion, and/or approximate a function.

“Opaque” generally refers to a material and/or article that has the physical property of blocking light or other forms of electromagnetic radiation from passing through the material. The material can be in the form of a solid, liquid, or gas. An opaque material is neither transparent nor translucent. Whether a material is opaque typically depends on the wavelength of the light and the nature of the material. For instance, some kinds of glass, while transparent in the visible light range, are largely opaque to ultraviolet light.

“Processor” generally refers to one or more electronic components configured to operate as a single unit configured or programmed to process input to generate an output. Alternatively, when of a multi-component form, a processor may have one or more components located remotely relative to the others. One or more components of each processor may be of the electronic variety defining digital circuitry, analog circuitry, or both. In one example, each processor is of a conventional, integrated circuit microprocessor arrangement. The concept of a “processor” is not limited to a single physical logic circuit or package of circuits but includes one or more such circuits or circuit packages possibly contained within or across multiple computers in numerous physical locations. In a virtual computing environment, an unknown number of physical processors may be actively processing data, and the unknown number may automatically change over time as well. The concept of a “processor” includes a device configured or programmed to make threshold comparisons, rules comparisons, calculations, or perform logical operations applying a rule to data yielding a logical result (e.g., “true” or “false”). Processing activities may occur in multiple single processors on separate servers, on multiple processors in a single server with separate processors, or on multiple processors physically remote from one another in separate computing devices.

“Reagent” generally refers to any substance that is added to a system that causes a chemical reaction. In other words, the reagent is any substance that is added to a system to cause a change in the chemical state of the system. The substance forming the reagent is normally consumed during the chemical reaction that the substance triggers. By way of non-limiting examples, this substance can include acids, bases, salts, and/or organic compounds, to name just a few examples. Reagents can be used in a wide variety of ways, including (but not limited to) to analyze chemical compositions, synthesize new compounds, purify substances, separate out components of mixtures, and/or change the rate of a chemical reaction. For example, reagents can be used in biology and medical diagnostics to identify and/or quantify specific medical conditions, like diabetes, elevated cholesterol levels, cancer, and the like. As another example, reagents can be used in analytical chemistry to identify and quantify unknown substances. Reagents can be for instance also used in organic synthesis to create new organic molecules. Reagents can further be used in material science to create new materials. Reagents can be categorized as primary reagents, secondary reagents, limiting reagents, and excess reagents. Primary reagents are the main reactant in a chemical reaction, and secondary reagents assist the primary reagents in carrying out the chemical reaction. Limiting reagents are consumed completely during the chemical reaction so as to set the maximum amount of product that can be formed, and excess reagents are present in larger quantities than the limiting reagents so that the excess reagents are not completely consumed by the chemical reaction.

“Recurrent Neural Network” or “RNN” generally refers to an artificial neural network wherein the outputs of one or more nodes affects one or more inputs to the same nodes. Some RNN's include memory to store various internal states and/or inputs. The memory of an RNN is stored within the RNN, stored in another neural network, and/or stored by another means. Typically, RNN's are used to perform tasks with inputs that have varying lengths. As examples, RNN's are used for identifying handwritten words, recognizing speech, generating speech, identifying actions in a video, predicting motion of an object, and/or performing other tasks. A neural network is considered fully recurrent if the outputs of all neurons in the network connect to the inputs of all neurons.

“Slide” generally refers to a thin piece of fully or mostly transparent material, like glass, quartz, or plastic, that supports one or more objects for visual examination such as under a microscope. An example of a standard microscope slide is a flat, rectangular piece of glass having the dimensions of 75 mm by 26 mm with a thickness of about 1 mm, but slides in other examples can be shaped and dimensioned differently as well as can be made from other materials. The slide is typically transparent or clear, but some parts of the slide may contain translucent or even opaque sections. For instance, the slide can be frosted or coated with enamel to facilitate labelling and/or writing on the slide. Graticule slides, for example, are typically marked with grid lines to facilitate counting and/or sizing objects on the slide such as for cell counting. The slide may further have a special coating such as to enhance chemical inertness and/or promote cell adhesion. While slides are normally flat, some slides may contain shallow depressions or wells, such as in the case of concavity slides or cavity slides, that hold a specimen or other object in place. Often, the object, such as a biological specimen, is held in place using a small transparent cover like a glass cover slip. The specimen can be mounted on the slide in several ways such as using dry mount, wet mount, prepared mount, and/or strewn mount techniques.

“Specimen” as well as “Biological Sample” or “Tissue Sample” generally refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

“Stain” or “Staining” generally refers to any treatment of a biological specimen that detects and/or differentiates the presence, location, and/or amount (such as concentration) of a particular molecule (such as a lipid, protein or nucleic acid) or particular structure (such as a normal or malignant cell, cytosol, nucleus, Golgi apparatus, or cytoskeleton) in the biological specimen. For example, staining can provide contrast between a particular molecule or a particular cellular structure and surrounding portions of a biological specimen, and the intensity of the staining can provide a measure of the amount of a particular molecule in the specimen. Staining can be used to aid in the viewing of molecules, cellular structures and organisms not only with bright-field microscopes, but also with other viewing tools, such as phase contrast microscopes, electron microscopes, and fluorescence microscopes. Some staining performed by the system can be used to visualize an outline of a cell or morphological detail. Other staining performed by the system may rely on certain cell components (such as molecules or structures) being stained without or with relatively little staining of other cell components. Examples of types of staining methods performed by the system include, without limitation, histochemical methods, immunohistochemical methods, special staining (e.g., Congo red, Trichrome, PAS, etc.), and other methods based on reactions between molecules (including non-covalent binding interactions), such as hybridization reactions between nucleic acid molecules. Particular staining methods include, but are not limited to, primary staining methods (e.g., H&E staining, Pap staining, etc.), enzyme-linked immunohistochemical methods, and in situ RNA and DNA hybridization methods, such as fluorescence in situ hybridization (FISH).

“Text” generally refers to one or more letters or similar symbols that form words to provide information.

“Translucent” generally refers to a material and/or article that has the physical property of allowing light or other forms of electromagnetic radiation to pass through the material but appreciably scatters the light so that objects beyond cannot be seen clearly. The material can be in the form of a solid, liquid, or gas. A transparent material is generally made up of components with different indices of refraction. Whether a material is translucent typically depends on the wavelength of the light and the nature of the material. Some examples of translucent materials include some forms of glass and plastics.

“Transparent” generally refers to a material and/or article that has the physical property of allowing light or other forms of electromagnetic radiation to pass through the material without appreciable scattering of light. The material can be in the form of a solid, liquid, or gas. A transparent material is generally made up of components with a uniform index of refraction. Transparent materials appear clear, with the overall appearance of one color, or any combination leading up to a brilliant spectrum of every color. Whether a material is transparent typically depends on the wavelength of the light and the nature of the material. Some examples of transparent materials include some forms of glass and plastics as well as air and liquid water.

“Vertical” generally refers to a plane and/or direction, which is perpendicular to the plane of the horizon. In another example, vertical is an alignment where the top is directly above the bottom. An item that moves upward or downward is generally said to move vertically. For example, an item that is able to move up and down is said to move vertically. In another example, the slope of a vertical line is undefined.

It should be noted that the singular forms “a,” “an,” “the,” and the like as used in the description and/or the claims include the plural forms unless expressly discussed otherwise. For example, if the specification and/or claims refer to “a device” or “the device”, it includes one or more of such devices.

It should be noted that directional terms, such as “up,” “down,” “top,” “bottom,” “lateral,” “longitudinal,” “radial,” “circumferential,” “horizontal,” “vertical,” etc., are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by the following claims are desired to be protected. All publications, patents, and patent applications cited in this specification are herein incorporated by reference as if each individual publication, patent, or patent application were specifically and individually indicated to be incorporated by reference and set forth in its entirety herein.

Reference Numbers

| | |
|---|---|
| 100 | system |
| 105 | slide |
| 110 | monitoring system |
| 112 | computer |
| 115 | interface |
| 120 | sample |
| 125 | imaging device |
| 130 | staining device |
| 135 | controller |
| 140 | camera |
| 145 | dispenser |
| 150 | reagent |
| 155 | memory |
| 160 | processor |
| 205 | substrate |
| 210 | work area |
| 215 | label area |
| 220 | system control area |
| 225 | tissue placement area |
| 230 | barcode |
| 235 | tissue control area |
| 240 | hydrophobic barrier |
| 300 | scanned image |
| 305 | control material |
| 310 | horizontal axis |
| 315 | slide information text |
| 320 | scanned stain |
| 325 | partial staining alert |
| 400 | vertical axis |
| 700 | results window |
| 705 | system control area size data |
| 710 | stain intensity data |
| 800 | histogram |
| 900 | histogram |
| 1000 | flowchart |
| 1005 | stage |
| 1010 | stage |
| 1015 | stage |
| 1020 | stage |
| 1025 | stage |
| 1100 | experimental results diagram |
| 1101 | first slide |
| 1102 | second slide |
| 1105 | first area |
| 1110 | second area |
| 1115 | third area |
| 1120 | fourth area |
| 1125 | fifth area |
| 1130 | sixth area |
| 1135 | seventh area |
| 1140 | eighth area |
| 1145 | ninth area |
| 1150 | tenth area |
| 1155 | eleventh area |
| 1160 | twelfth area |
| 1162 | graph |
| 1165 | density-axis |
| 1170 | concentration-axis |
| 1175 | density-concentration line |
| 1300 | graph |
| 1310 | time axis |
| 1320 | control property axis |
| 1330 | fluidic coverage data |
| 1340 | intra-run line |
| 1350 | inter-run line |
| 1360 | gap |
| 1370 | fluidic failure point |
| 1400 | slide |
| 1405 | dual detection system |
| 1410 | first control |
| 1420 | second control |
| 1500 | slide |
| 1510 | scanned image |
| 1520 | processed image |
| 1530 | system control dot |
| 1600 | chart |
| 1700 | chart |
| 1800 | chart |
| 1900 | slide |
| 1910 | first control dot |
| 1920 | second control dot |
| 2000 | chart |

What is claimed is:

1. A slide, comprising:
a work area having one or more system control areas;
wherein the system control areas have one or more control materials; and
wherein the control materials are configured to facilitate monitoring of assay procedures.

2. The slide of claim 1, wherein:
the work area has corners; and
the system control areas are positioned at the corners of the work area.

3. The slide of claim 2, wherein the work area has a rectangular shape.

4. The slide of claim 1, further comprising:
one or more hydrophobic barriers being configured to contain fluids on the slide.

5. The slide of claim 1, further including:
a label area having a barcode.

6. The slide of claim 5, further comprising:
one or more hydrophobic barriers positioned between the work area and the label area.

7. The slide of claim 1, wherein the control materials are tissue samples.

8. The slide of claim 1, wherein the control materials include specific tumor types.

9. The slide of claim 1, wherein the control materials include detection antibodies.

10. The slide of claim 1, wherein the control materials are covered with paraffin.

11. The slide of claim 1, wherein the work area has a tissue placement area.

12. The slide of claim 1, wherein the work area has a tissue control area configured to receive a tissue.

13. The slide of claim 1, wherein at least one of the system control areas includes a dual detection system.

14. The slide of claim 13, wherein:
the dual detection system includes a first control and a second control; and
the first control and the second control are different.

15. The slide of claim 13, wherein the dual detection system is configured to indicate that the slide is fully covered by a staining fluid.

16. The slide of claim 13, wherein the dual detection system is configured to facilitate detection of false negatives.

17. The slide of claim 1, wherein the slide has a background color, and the background color provides a high contrast for the system control areas.

18. A system, comprising:
a monitoring system including at least one imaging device;
wherein the imaging device is positioned to capture one or more images of a slide;
the slide having a work area;
the work area having one or more system control areas;
wherein the monitoring system includes a controller;
wherein the controller is operatively coupled to the imaging device; and
wherein the controller is configured to determine if the slide has been properly stained based on the images of the system control areas of the slide.

19. The system of claim 18, further comprising:
a dispenser configured to dispense fluid onto the slide; and
wherein the controller is operatively coupled to the dispenser.

20. The system of claim 19, wherein:
the fluid includes a reagent; and
the controller is configured to determine if the work area has been covered with the reagent.

21. The system of claim 19, wherein the monitoring system is configured to monitor area size of the system control areas that react with the fluid.

22. The system of claim 19, wherein the monitoring system is configured to monitor intensity of the system control areas that react with the fluid.

23. The system of claim 19, wherein the monitoring system is configured to monitor density of the system control areas.

24. The system of claim 18, further comprising:
a computer being operatively coupled to the controller;
wherein the computer includes an interface; and
wherein the interface is configured to provide processing information concerning the slide.

25. The system of claim 18, wherein the controller is configured to track intra-run variability.

26. The system of claim 18, wherein the controller is configured to track inter-run variability.

27. The system of claim 18, wherein the controller is configured to detect fluidic failures.

28. The system of claim 18, wherein the system control areas include at least two standardized substances that are different.

29. A method, comprising:
capturing one or more images of one or more system control areas of a slide with a camera of a monitoring system;
determining the slide has not been processed in a correct manner with a controller of the monitoring system; and
providing feedback in response to the determining the slide has not been processed in the correct manner with the monitoring system.

30. The method of claim 29, wherein:
the system control areas have one or more control materials; and
the control materials are configured to facilitate monitoring of assay procedures.

31. The method of claim 29, further comprising:
tracking intra-run variability with the monitoring system.

32. The method of claim 29, further comprising:
tracking inter-run variability with the monitoring system.

33. The method of claim 29, further comprising:
determining the slide has not been processed in the correct manner includes detecting fluidic failures.

34. The method of claim 29, further comprising:
monitoring for cytology staining with a dual detection system.

35. The method of claim 29, further including:
detecting false negatives with a dual detection system.
